(19) United States
(12) Patent Application Publication
(10) Pub. No.: US 2025/0062016 A1
(43) Pub. Date: Feb. 20, 2025

(54) METHOD OF ESTABLISHING A DECISION SUPPORT ENGINE FOR MEDICAL EQUIPMENT

(71) Applicant: EquipX, LLC, Aventura, FL (US)

(72) Inventor: Ilan Mintz, Aventura, FL (US)

(21) Appl. No.: 18/936,843

(22) Filed: Nov. 4, 2024

Related U.S. Application Data
(63) Continuation of application No. 17/127,185, filed on Dec. 18, 2020.
Application No. 17/127,185 is a continuation-in-part of application No. 17/000,267, filed on Aug. 21, 2020, now abandoned.
(60) Provisional application No. 62/889,966, filed on Aug. 21, 2019.

(51) Int. Cl. G16H 40/20; G06Q 30/02

(52) U.S. Cl. CPC G16H 40/20; G06Q 30/0278

(57) ABSTRACT

A decision support engine provides a table of actual and predicted financial results particular to a selected medical equipment. A database stores parameter data of various medical equipment belonging to the organization. The stored parameters may include purchase date and price. An appraisal module uses the parameter data to generate an appraised value of each of the medical equipment. Upon selection by a user of a specific equipment, the decision support module provides past period financial results attributable to the selected equipment, and predicted financial results that may be achieved upon upgrade or replacement of the equipment by newly purchased or leased equipment. The entire system may reside in the cloud.

### RELATED APPLICATION

This application is a continuation of U.S. application Ser. No. 17/127,185, filed Dec. 18, 2020, which is a Continuation in Part of U.S. application Ser. No. 17/000,267, filed Aug. 21, 2020, which claims the benefit of U.S. Provisional Application No. 62/889,966, filed Aug. 21, 2019, which are hereby incorporated by reference in its entirety.

### FIELD

The present disclosure generally relates to the medical field and, more particularly, to evaluation of medical equipment and making decisions regarding maintenance, upgrades and replacement.

### DESCRIPTION OF THE RELATED ART

Pre-owned or used medical equipment is an often-overlooked asset in a hospital or medical practice. Generally, decisions regarding capital spending on medical equipment are normally made by consensus of a committee, based on historical trends, rather than forecasting for future needs. The medical equipment of a medical facility is generally counted as an expense, and its value is indicated only by its depreciated value in the accounting ledger, rather than its value in the used market place.

Most health systems do not know the operating costs, revenue, utilization and profitability of their capital equipment. The lack of metrics (e.g., profit and loss) for capital equipment means that clinical service line leaders have no visibility into the contribution they make to the health system's profitability. The depreciated book values of a health system's most expensive equipment typically represent only 70% of current market value.

Moreover, managers have no data with which to make a decision relating to any of the capital equipment in their healthcare system. For example, a specific machine may require much maintenance, which reduces its availability, thus reducing its utilization. Consequently, the equipment may be operating at a loss, without managers having any data to be alerted to such a situation. Additionally, even when the increased maintenance costs are detected, there is no information to a manager to decide which action would provide a better financial return, a refurbishment, an upgrade, a replacement, a lease, etc.

What is needed is a system and method for evaluating medical equipment enabling better evaluation of equipment utilization efficiency and forecast-based purchasing and selling decisions.

### SUMMARY

The following summary of the disclosure is included in order to provide a basic understanding of some aspects and features of the invention. This summary is not an extensive overview of the invention and as such it is not intended to particularly identify key or critical elements of the invention or to delineate the scope of the invention. Its sole purpose is to present some concepts of the invention in a simplified form as a prelude to the more detailed description that is presented below.

Embodiments disclosed herein provide financial and operational visibility to a healthcare organization regarding each individual asset employed in the organization. Disclosed embodiments also provide complete transparency into capital equipment performance throughout its entire lifecycle. Employing the disclosed embodiments improves forecasting of capital needs based on predictive analytics. Moreover, by providing transparency into the equipment performance, better decisions regarding replacement can be made.

Disclosed embodiments provide systems and methods for evaluating medical equipment. Aspects of disclosed embodiments provide solutions for hospitals to appraise, value, negotiate, resell and deliver their preowned or excess medical equipment to a wide array of buyers on a global platform.

Disclosed aspects include a system for determining market value of medical equipment, a computer-implemented method for determining market value of medical equipment, and a non-transitory machine-readable medium having instructions stored therein, which when executed by a processor, cause the processor to estimate market value of a medical equipment.

Disclosed embodiments may provide estimated trade value of each particular medical equipment, to be used by organizations such as hospitals and clinics considering purchasing or selling equipment, leasing companies that may be interested in determining residual value of each equipment, service and outsourcing companies interested in, e.g., pricing their services, group purchasing organization, etc.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 illustrates an overview of a marketplace screenshot, in accordance with one embodiment of the present invention.

FIG. 2 illustrates lifecycle optimization platform in accordance with one embodiment of the present invention.

FIG. 3 illustrates a graph of a value optimization model, in accordance with one embodiment of the present invention.

FIG. 4 illustrates a seller valuation range examples screenshot, in accordance with one embodiment of the present invention, in accordance with one embodiment of the present invention.

FIG. 5 illustrates a seller price gauge display screenshot, in accordance with one embodiment of the present invention.

FIG. 6 is a graph illustrating the use of the market value estimator to enable determination of equipment disposition and arbitrage opportunity, according to an embodiment.

FIG. 7 illustrates a snapshot of a dashboard, including a representation of inputs, according to an embodiment.

FIG. 8 is a general schematic illustrating the major components of the system according to an embodiment.

FIG. 9 is another general schematic illustrating the major components of the system according to an embodiment.

FIG. 10 is a flow chart illustrating a method that may be performed by the system to calculate profitability of a medical equipment, according to an embodiment.

FIG. 11 is a schematic illustrating the integration feature of the system, according to an embodiment.

FIG. 12 is a screenshot of a user dashboard according to an embodiment.

FIG. 13 is a block diagram illustrating an arrangement for a decision support module according to an embodiment.

FIG. 14 is an example of a user dashboard for decision support according to an embodiment.

FIG. 15 is a flow chart illustrating an example of a process that may be used for the dashboard of FIG. 14, according to an embodiment.

FIG. 16 is a flow chart illustrating an example of a process that may be used in conjunction with the dashboard of FIG. 14, according to an embodiment.

FIG. 17 illustrates an example for a decision table aggregating operational data of medical equipment corresponding to a user-selected modality, according to an embodiment.

### DETAILED DESCRIPTION

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention however the order of description should not be construed as to imply that these operations are necessarily order dependent. In particular, these operations need not be performed in the order of presentation. Moreover, different features may be highlighted in different embodiments, but should not be construed as limited only to the embodiment within which they are disclosed. Indeed, the features may be “mixed and matched” with different embodiments, as one finds different benefits.

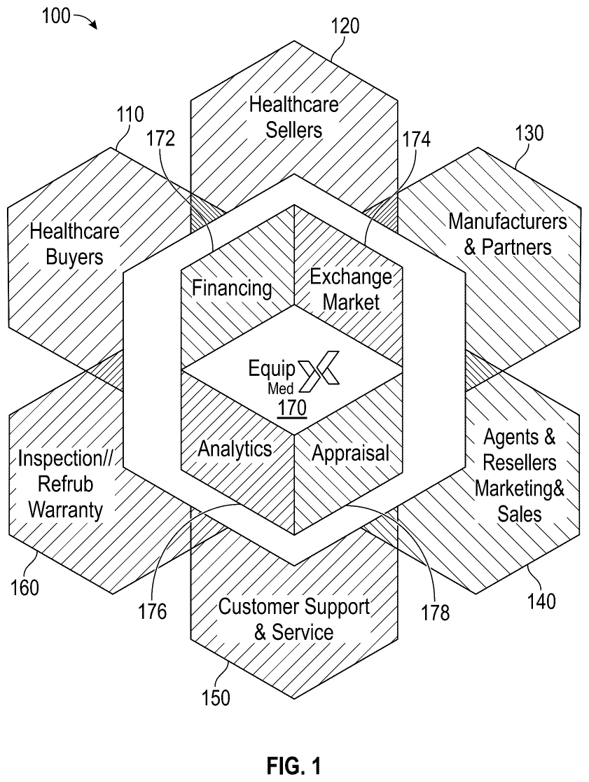

FIG. 1 illustrates an overview of a marketplace 100 for medical equipment, in accordance with one embodiment of the present invention. The system 170 operates within the marketplace 100 using a plurality of modules, and provides access to a plurality of different entities. The entities may include a plurality of healthcare buyers 110, a plurality of healthcare sellers 120, a plurality of manufacturers and partners 130, a plurality of agents, resellers, marketing and salespersons 140, a customer support and service 150, an inspection and repair warranty 160. The overall system 170 may include a plurality of modules, including financing module 172, an exchange market module 174, analytics module 176 and an appraisal module 178. The modules operate to provide the entities analysis based on data input, which helps in making capital equipment decisions.

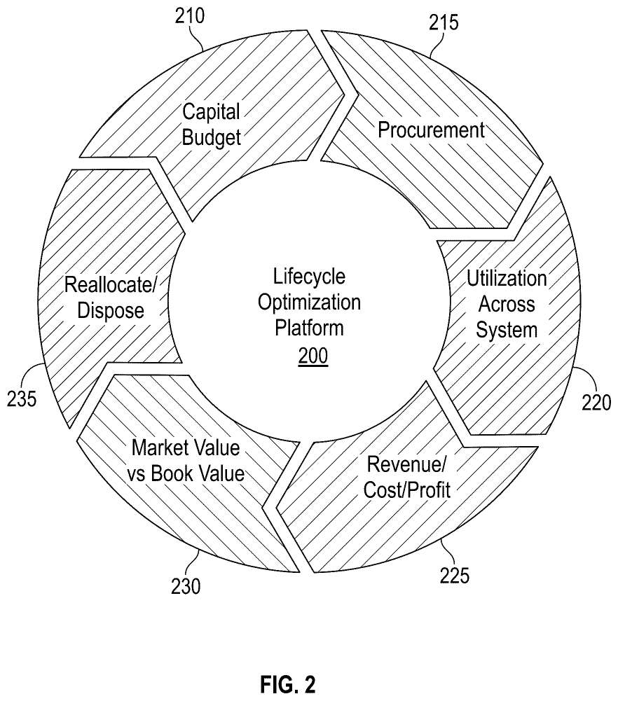

FIG. 2 illustrates an embodiment of a lifecycle optimization platform. As the lifecycle is circular, the processing may begin at any part of the cycle, but for illustration this description begins at the capital budget module 210. The capital budget module provides accurate use projection for each capital equipment, including financial performance data. The capital budget module includes a budget simulator module that enables a user to simulate various scenarios using decision entries such as: reallocation or relocation of the equipment, sale of the equipment, and performing life extending maintenance. The procurement support module 215 includes an equipment valuation calculator, which will be described in more details below. The procurement support module 215 also includes a return on investment (ROI) calculator and a market sale price module. The utilization module 220 includes utilization calculator that provides actual utilization of each capital equipment deployed in the system. The utilization module also provides use comparison of various equipment and use trends by equipment type.

The revenue module 225 includes revenue calculator that provides real time revenue generated by each deployed asset. The revenue module 225 also provides the operating cost of each asset and profitability by asset and category of assets. Further, the profit margins are calculated at the equipment, department and procedure levels. The value module 230 provides an analysis of market versus book value of each asset. It includes market price estimator and generates an optimal point of sale indicator. The disposition module 235 also employs the market value estimator and generates a trade-in comparison. The disposition module also includes a financial simulator which enables calculating outcomes for different disposition scenarios. Additionally, the disposition module also incorporates a marketplace for online purchase and sell of medical equipment.

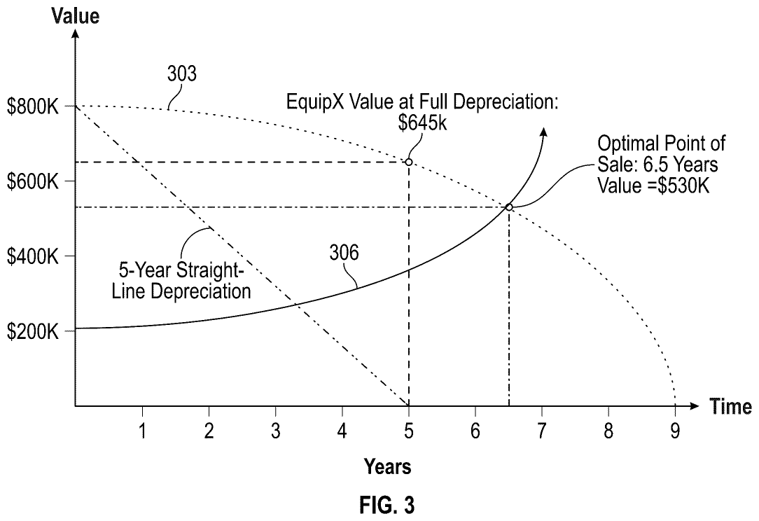

FIG. 3 illustrates a graph of a value optimization model, in accordance with one embodiment of the present invention. The value optimization model may include a time X-axis and a money value Y-axis. In this embodiment a straight line depreciation is used, illustrated by the straight line from $800 K value (arbitrarily chosen) and ending at 5 years. The market value estimator 230 (to be described below) is used to generate estimated market value over time and generate the down-sloping curve 303. As can be seen, the calculated market value differs from the book value that is indicated by the depreciation line. Additionally, revenue module 225 calculates the operating costs of the equipment, which is illustrated by up-sloping curve 306. According to this example, the optimal point of sale is defined as the intersection of the market value curve 303 and operating cost curve 306. This may be considered as an arbitrage point, as from accounting perspective, the organization owns a fully depreciated assets, which has zero value on the books. However, the market value of the equipment is much higher, so that in effect the system of this embodiment uncovered previously unrecognized value within the organization.

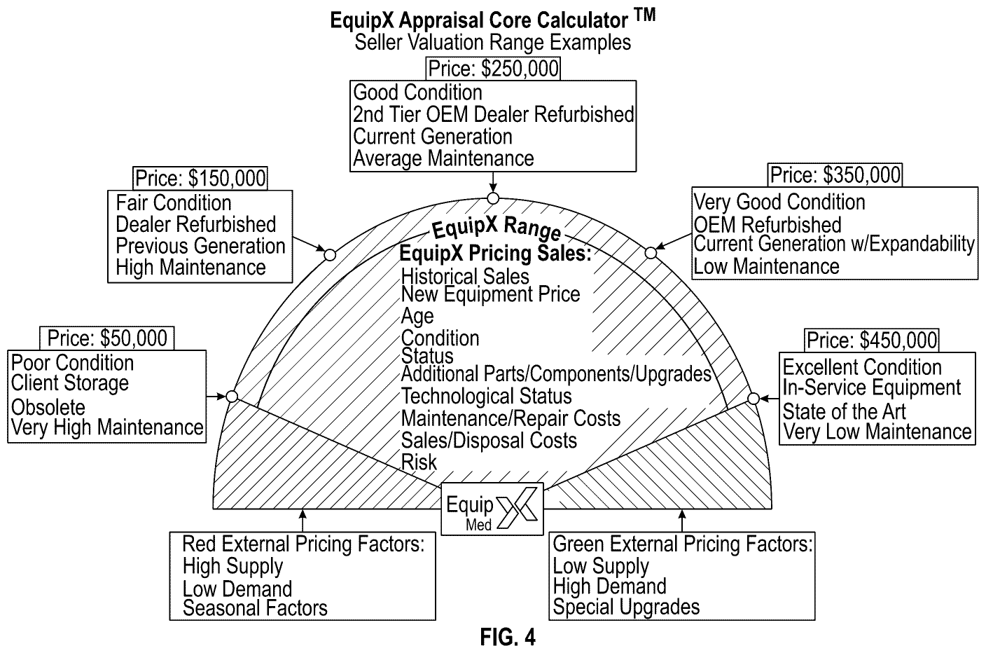

FIG. 4 illustrates a graph of seller valuation range screenshot, in accordance with one embodiment of the present invention. The example of FIG. 4 illustrates the operation and benefits of the market value estimator 230. The market value estimator receives data regarding various factors, assigns weights to each factor, and calculate a market price therefrom. The factors may include historical sales prices, the new equipment price, the age of the equipment, the condition of the equipment (as selected by the user), the status of the equipment (operational, in storage, undergoing refurbishment, etc.), additional parts components and/or upgrades conveyed with the equipment, technological status of the equipment, maintenance costs, disposition costs, and risk factors. The pricing examples illustrated in FIG. 4 are fictitious and used for illustration only. The values generated by the market value estimator 230 may be used to generate graph 306 shown in FIG. 3.

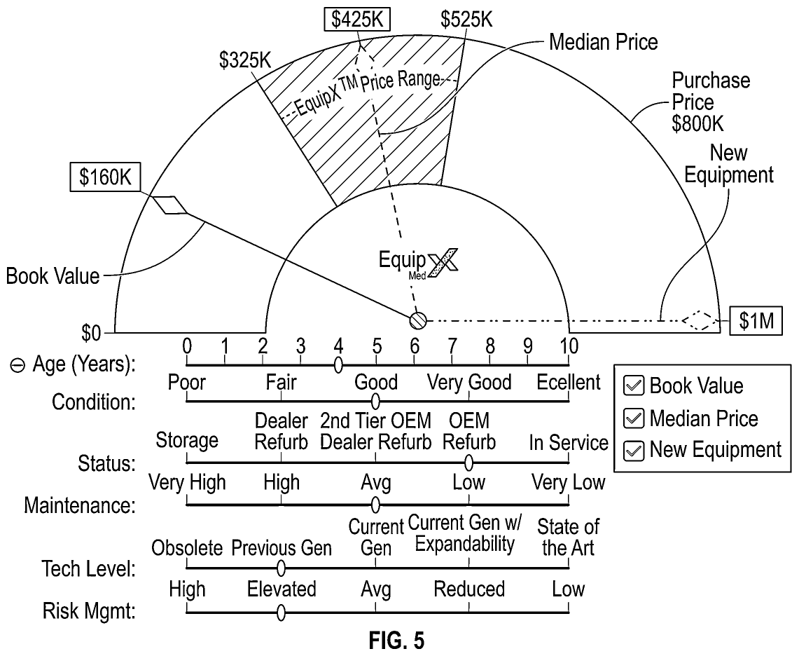

FIG. 5 illustrates a seller price gauge display screenshot, in accordance with one embodiment of the present invention. The seller price gauge display enables a potential seller to enter various factors and generate an estimated market price using the market value estimator 230. The seller price gauge display also enables the user to see how different factors may affect the estimated market price, so as to make decision regarding, e.g., maintenance, upgrades, refurbishment, and sell of the equipment.

In this example, discrete values are provided for each factor for the user to select. Once these discrete values are selected, the market value estimator 230 applies variable weights to each of the factors and then calculates the market value. In this example the factors include age, condition, status, maintenance, technology level, and risk. In the example of FIG. 5 the market value price is displayed in the form of a semi-circular speedometer style gauges, with a first dial indicating the current book value of the equipment and a second dial indicating the calculated market value of the equipment. Whenever the calculated market value of the equipment is to the right, i.e., higher than the book value dial, an arbitrage opportunity exists.

Also, in FIG. 5 the factors are presented as digital slides (selectors or dials) having discrete stations for the user to select presented values by positioning the slides or dials on the selected values. In this manner, as the user changes any selected value, the user is able to immediately see the movement of the dial on the speedometer to understand the effect of that particular selection on the market value calculation. FIG. 5 may also include a third dial which indicates the new equipment price. The user may be provided with check boxes to decide which dials to display on the speedometer.

In disclosed embodiments, the market value estimator 230 incorporates various factors and determines weights to each individual factor as follows. The actual historical sales of fundamentally similar equipment may be a factor in the calculation. In disclosed embodiments the weight applied to the historical sales is variable, and increases as the volume of sales data collected increases. Notably, for each type or model of equipment different amount of data of actual sales may be available. Thus, the weight is assigned specific to the individual equipment.

If the equipment/model under consideration is still in production, the prices of new, replacement equipment will be factored into the market value calculation. If the price of new equipment has increased over time, this will have a positive impact on the price of the Seller's equipment. If the price of new equipment has decreased over time, this will have a negative impact on the price of the Seller's equipment. A percentage scale affecting the market value price will be applied to the equipment based upon the price of new/comparable equipment. In one embodiment the weights may be −15%; −10%; −5%; 0%; 5%; 10%; 15%.

The equipment's age may be a factor in the market value calculation. The newer the equipment, the higher the market value price. Actual age (in years) may be used as a factor in the market value calculation, and an applied weight may be changed to reflect the age. For example, higher weight at the edges (newer or very old equipment, and lower weight at the center reflecting average age).

Another factor may be equipment status. Equipment in working condition will be assigned a higher weight than equipment in non-working condition. Unused equipment in storage will be assigned a weight based upon its status and length of time in storage. A discrete scale may be applied, for example: 1=equipment in client storage; 2=dealer refurbished equipment; 3=2nd tier OEM dealer refurbished equipment; 4=OEM refurbished equipment; 5=fully functional in-service equipment.

A further factor may be the equipment's condition, as selected by the user. That is, the user would be provided a discrete scale selection and the input from the user would be used for the weight. The equipment will be assigned a factor based on its condition, e.g., using the scale: 1=Poor; 2=Fair; 3=Good; 4=Very Good; 5=Excellent.

Another factor may be the technical status of the equipment. Obsolete or previous generation equipment will have a limited test menu. Current generation or state of the art equipment will have an expanded test menu. The equipment will be assigned a factor based on its technological status using a scale, e.g., 1=Obsolete; 2=Previous Generation; 3=Current Generation; 4=Current Generation with Expandability; 5=State of the Art.

Maintenance and repair costs may be considered as a percentage of the cost of the equipment. The factor increases inversely with the maintenance cost, i.e., the less it costs to maintain, the higher the factor value. This may also be assigned on a scale of discrete values, e.g., 1=Very High Maintenance Costs; 2=High Maintenance Costs; 3=Average Maintenance Costs; 4=Low Maintenance Costs; 5=Very Low Maintenance Costs.

Some equipment may include additional parts or upgrades, some by the equipment manufacturer and some by non-OEM aftermarket suppliers. If a piece of equipment has manufacturer (OEM) upgrades or parts, it will result in an increase in the market value price. A non-OEM upgrades or parts may affect the market value price negatively.

Generally, sale or disposal of the equipment would incur transaction costs, which may be factored into the market value price. The disposal costs may be selected by the user using a discrete scale expressed as a percentage of the market value sale price.

Purchase of used equipment entails assuming risks. The risk may be scaled based on the selling situation. For example, an OEM certified equipment offered for sale by a major hospital may be assigned a low risk, while sale of equipment at an auction may be assigned a high risk. Again, the factors would be presented to the user as discrete values for selection, e.g., 1=High Risk; 2=Elevated Risk; 3=Average Risk; 4=Reduced Risk; 5=Low Risk.

In some embodiments, each of the factors are presented to the user as discrete values for selection. The entered values are then used with assigned weights to generate market value price using the expression:

$$Price = IP - α * A - β * S - δ * C - ε * T - ω * M - π * P ,$$

Wherein IP is the equipment initial price, A is the equipment age, S is the equipment status, C is the equipment condition, T is the technological status, M is the equipment maintenance costs factor (using scale values, not actual dollar costs), and P is additional parts factor value. The weight variables α, β, δ, ε, ω, π may be determined by historical data and are assigned different values depending on the equipment type/model. Also, in some embodiments the market value is provided as a range rather than a single value. The range is calculated as plus minus the market value price obtained from the expression provided above. That is, for an example where the percentage chosen is 5%, the range is from (price −5%) to (price +5%).

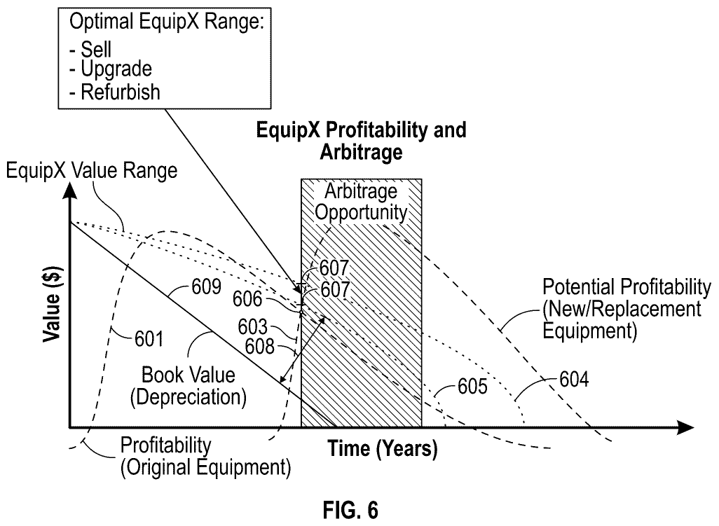

FIG. 6 is a graph illustrating the use of the market value estimator to enable determination of equipment disposition and arbitrage opportunity, according to an embodiment. In FIG. 6 the x-axis expresses time in years, while the y-axis expresses value, e.g., in dollars or relative scale. This graph shows the optimal time to sell or upgrade/refurbish the equipment. Curve 601 indicates the profitability of the equipment over time. Over time, the profitability will begin to decrease as equipment ages and maintenance costs increase. As noted above, the revenue module 225 can evaluate the potential profitability of new or upgraded/refurbished equipment indicated as curve 603 in dashed line. The market value calculator is then used to determine upper and lower estimates for the market value of the equipment. The system then identifies when is the best time to perform the sale/upgrade of the equipment in order to maximize profits. This is represented on the graph by the point 606 where the profitability curve and potential profitability curve cross each other. The price at which the equipment might be sold is indicated by the two points 607, where the sell time crosses the two market value range curves.

FIG. 6 also illustrates the arbitrage opportunity calculated by the system. The arbitrage opportunity is the spread (or area) between the book value curve 609 and the curves of the market value 604 and 605. This is indicated in FIG. 6 by the double-headed arrow 608. This value is not recognized by the institution as the accounting books only show the depreciated value. Note that the variability between the high and low market value curves increase over time due to changes in the market value pricing factors, e.g., equipment condition deteriorates, technological obsolescence increases, the price of new/comparable equipment changes, etc.

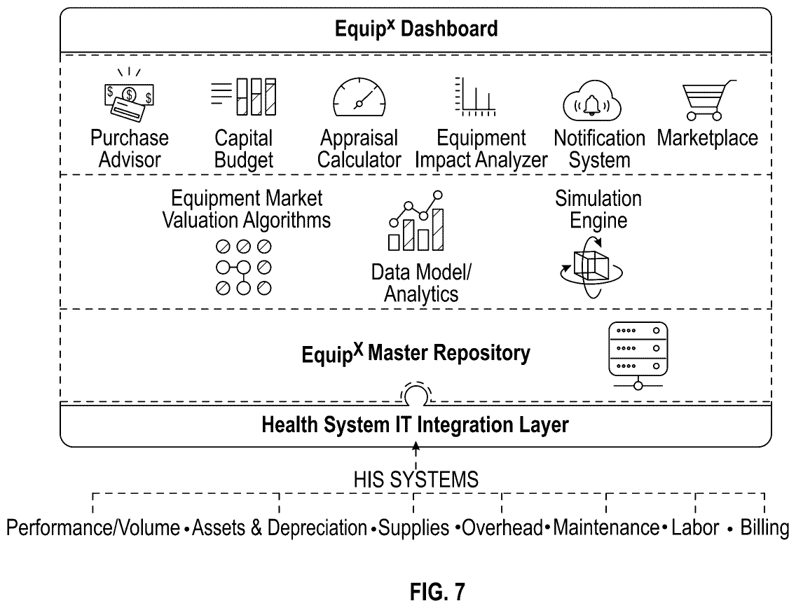

FIG. 7 illustrates a snapshot of a dashboard, including a representation of inputs, according to an embodiment. As shown in the bottom of FIG. 7, various inputs may be provided to the system to be used in various calculations. The inputs may include volume of procedures performed by the equipment, list of assets and depreciation values, costs of supplies used with the equipment, overhead costs associated with the equipment, maintenance cost of the equipment, labor costs associated with the equipment, and all billings associated with the equipment (to determine revenue attributed to the equipment). The input data is assigned to the various medical equipment deployed. The dashboard enables the user to activate the various modules of the system. The various modules may include a purchase advisor which may provide purchasing recommendations based upon, e.g., the output of the market value estimator, which is shown as appraisal calculator in FIG. 7. The equipment impact analysis module can be used to determine profitability of each equipment, the impact of an upgrade or refurbishment, etc. The marketplace module can be used to place equipment for sale or to purchase equipment.

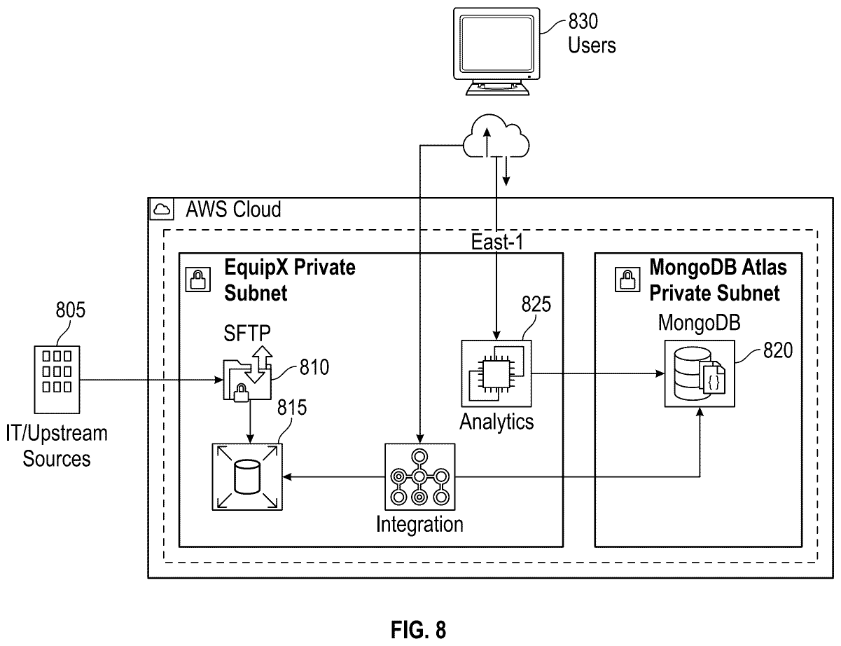

FIG. 8 is a general schematic illustrating the major components of the system according to an embodiment. Various outside sources 805 may upload data into the system. For example, the sources may include manufacturers uploading new equipment pricing, used equipment marketplace uploading actual sales data, service companies uploading maintenance price sheets, etc. The data may be sent to the cloud using the Secure File Transfer Protocol 810 to be analyzed and stored by data processor 815. All data, analytics, calculation results, etc., may be stored in the database 820. Analytics processor 825 performs the analysis to provide user 830 with the results and recommendations, as disclosed herein.

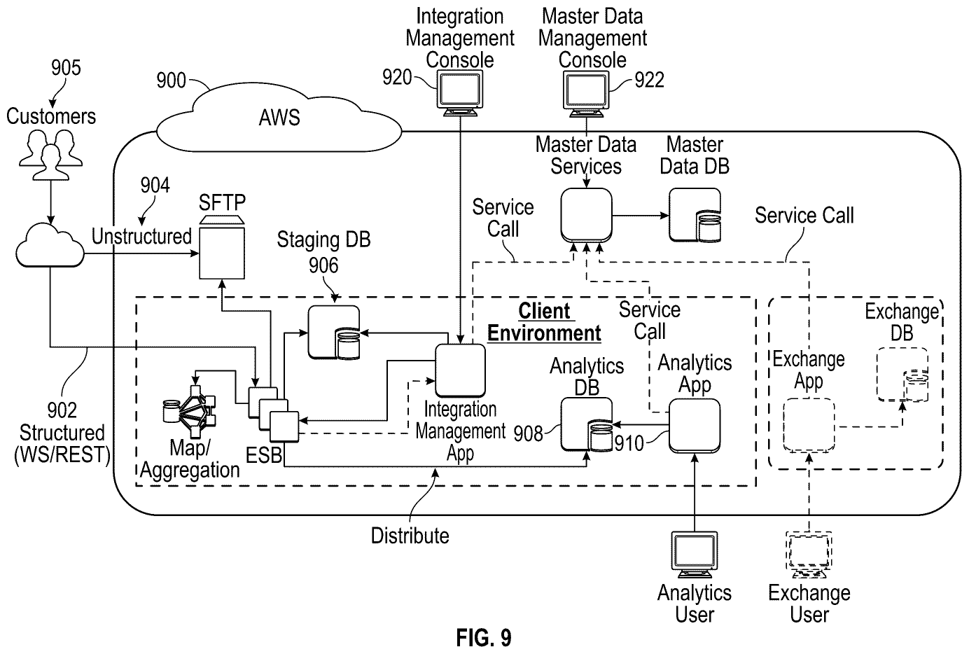

FIG. 9 is another general schematic illustrating the major components of the system according to an embodiment. The entire system may reside in the cloud 900 and be provided to users 905 as a software as a service (SaaS). Data may enter the system as structured 902 or unstructured 904 data. The data is organized and stored in staging database 906. Analytics modules 910 may access and store data in analytics database 908. The entire system is managed by integration management console 920 and master data management console 922.

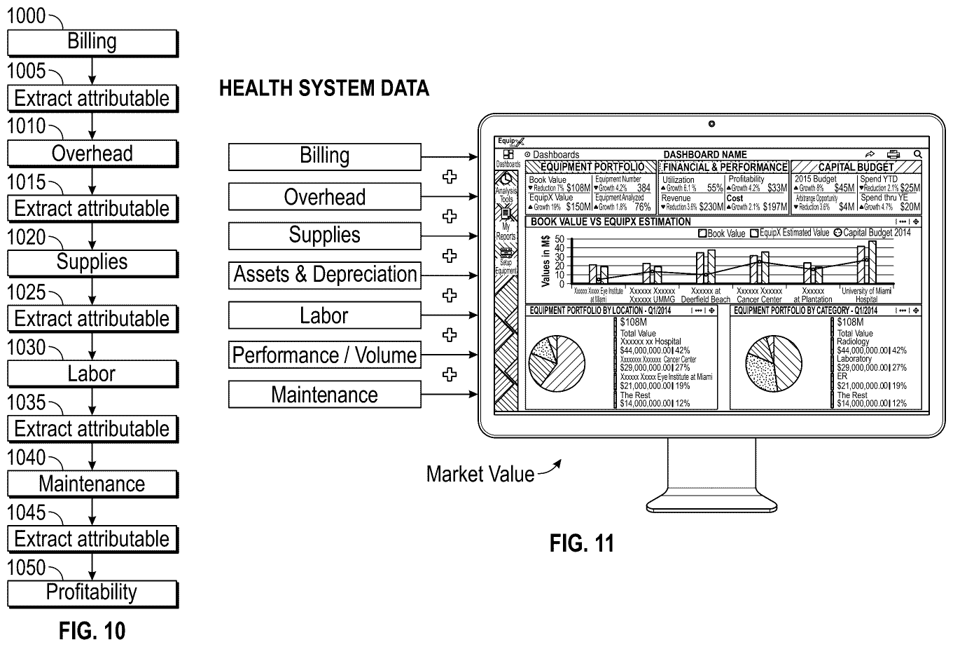

FIG. 10 is a flow chart illustrating a method that may be performed by the system to calculate profitability of a medical equipment, according to an embodiment. At 1000 the system receives billing information that includes bills sent out by the institution to bill for various medical services. At step 1005 the system extracts the amount of each bill which is attributable to the particular medical equipment. At 1010 the system receives data of the overhead expense of the facility. At 1015 the system extracts the overhead that is attributable to the operation of the medical equipment. For example, a lease payment for the entire facility cannot be applied directly to the particular equipment, but must be extracted to include only cost associate with the equipment, e.g., a ratio of size of the room where the equipment is placed to the size of the entire building. At 1020 the system receives data reflecting payments made for purchasing supplies. At 1025 the system utilizes its knowledge database to decipher the type of supplies used by the particular equipment to extract only the cost of supplies attributable to those supplies. At 1030 the system receives labor expense data. At 1035 the system utilizes its knowledge database to determine the type of labor that is attributable to the equipment. For example, labor costs of an x-ray technician may be attributable to an x-ray machine, but surgeons' labor expense may not be. At step 1040 the system receives maintenance data and at 1045 the system utilizes its knowledge base to extract only maintenance attributable to the specific equipment. Using all the extracted data, the system at 1050 can calculate the profitability of the particular equipment.

An example of the system integration is illustrated in FIG. 11. The system receives various data from the health care system and utilizes various modules to extract appropriate data for each particular equipment and therefrom generates an estimated trade value for each equipment, time to replace the equipment, residual value of leased equipment, potential fair market price for a group purchasing organizations (GPO), etc. The input may include billing information, overhead costs, supplies costs, assets and depreciation, labor costs, performance and utilization and maintenance costs. As described herein, the system extracts only the data relevant to a particular equipment and assigns to each individual medical equipment the portion attributable to that particular equipment.

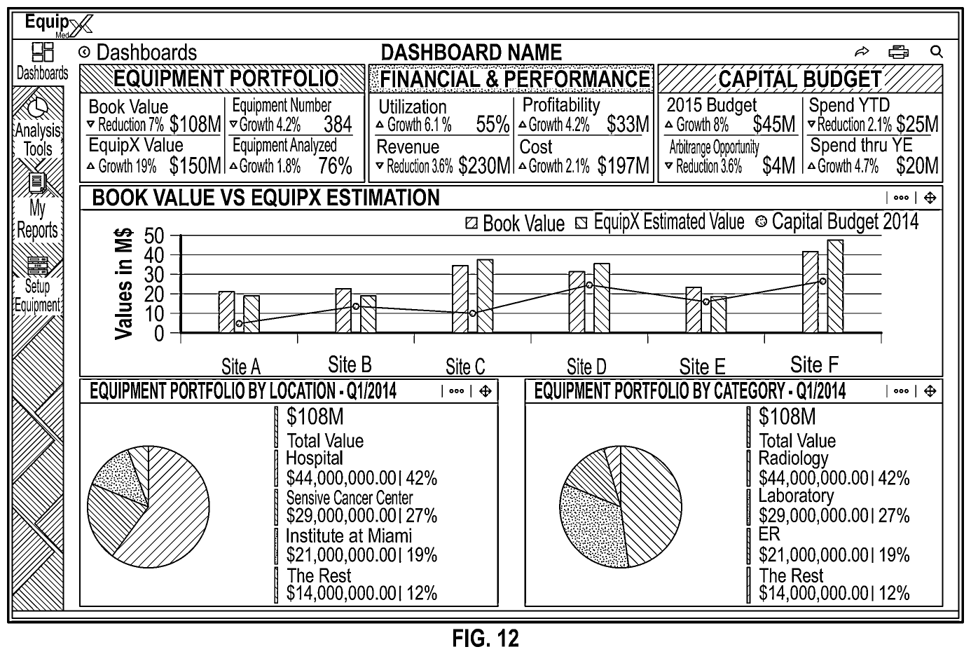

The system then displays a dashboard for the user to enable the user to understand the financial effect of each particular medical equipment on the performance of the entire organization, as illustrated in FIG. 12. As shown in FIG. 12, the system can be used for optimization of the utilization of the equipment. This may also be done using simulations of various scenarios for each equipment. The system may present data by assigning various equipment to different departments to determine the efficiency at each department. The data is also visualized by each medical center by aggregating all the equipment operating within that medical center.

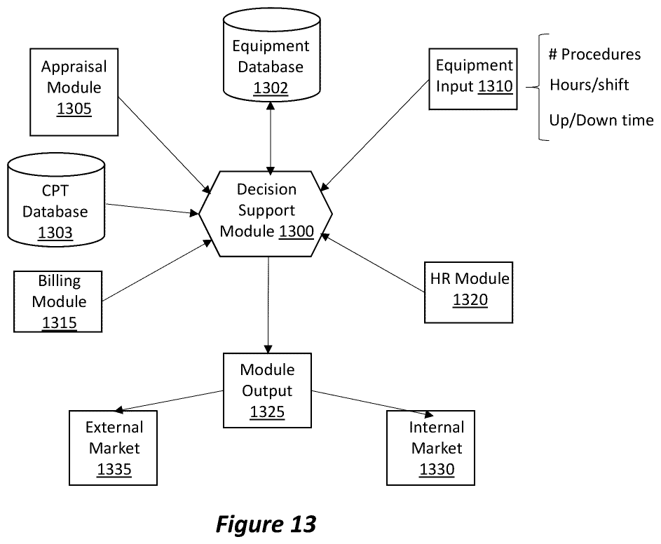

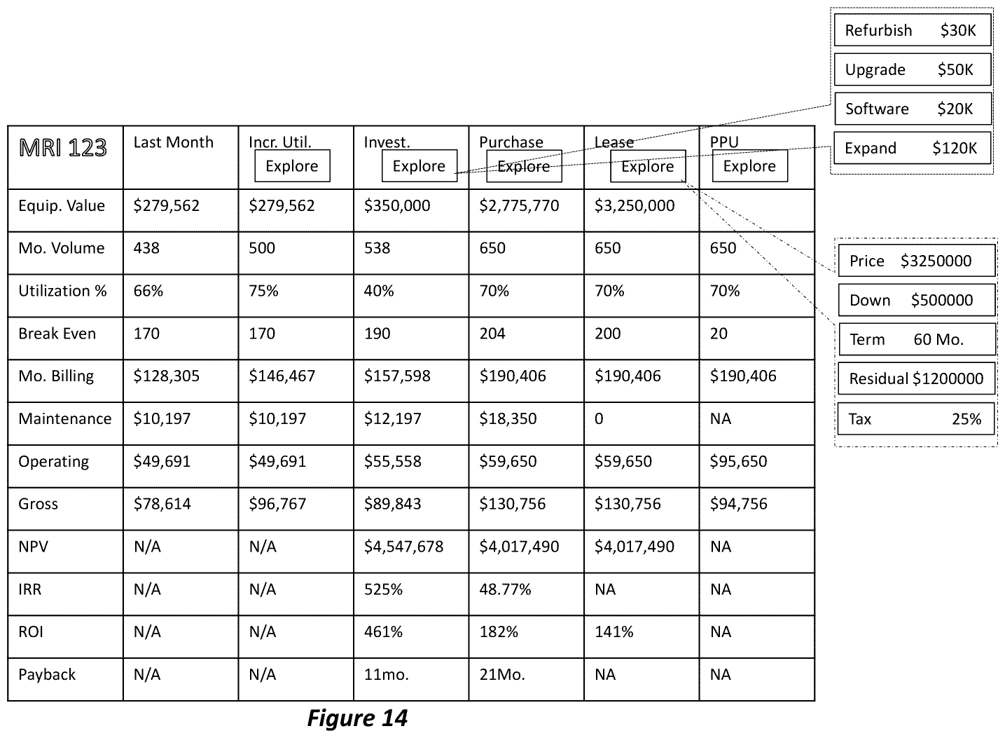

An important issue for managers of medical facilities is to understand the financial performance of each piece of equipment and determine whether the equipment should be refurbished, upgraded, or replaced by new equipment. With respect to replacement, it is also difficult to decide whether to purchase or lease the new equipment. Also, some vendors offer a “pay-per-use” type arrangement, whereby the vendor installs and supports the equipment, and the healthcare organization pays an agreed amount per procedure performed on the equipment. FIG. 13 illustrates a block diagram of a decision support module, interconnected with other system modules to provide decision support for each specific equipment, according to an embodiment. FIG. 14 illustrates an example of the user interface output of the module providing the user with financial performance and projections corresponding to chosen actions specific to each equipment. A similar procedure may be implemented to generate financial performance and projections per modality, e.g., all CT scanners within the organization.

The decision support module 1300 operates in conjunction with several other modules in order to gather the necessary data to generate the values for the presentation in FIG. 14, specific to a single selected equipment or modality, here indicated as MRI 123. In the example of FIG. 14, the first column includes historical performance values generated for prior period(s), in this example for the past month. The second column includes projected values generated as estimates resulting from changes of usage of the equipment, here increase in utilization of the equipment. Conversely, the third column includes projected values generated as estimates resulting from changes to the equipment itself, e.g., refurbishment, upgrade, etc. The fourth column includes values generated as estimates resulting from purchase of new equipment, while the fifth column includes values generated as estimates resulting from lease of new equipment. The optional sixth column includes values for a pay-per-use arrangement, wherein the organization does not invest in purchase or lease, but simply pays a vendor per procedures performed on the equipment. Note that in this context the vendor may be a financial company that purchases the equipment from the manufacturer and signs the pay-per-use agreement with the health organization. It should be noted that all of the values illustrated in FIG. 14 and in this example are fanciful and are used solely for illustrating the embodiment.

The first row includes estimated values corresponding to the calculated market value of the equipment. The appraisal module 1305, as detailed herein with respect to, e.g., FIGS. 3-5, provides an updated appraised value of the equipment calculated for the past period, here the last month. This value is received by the decision support module 1300 and is presented in the first and second columns. To generate the value in the third column, the proposed action (e.g., refurbishment, upgrade, software update, etc.) and its cost is sent by the decision support module 1300 to the appraisal module 1305 to use in recalculating the appraised value of the equipment after the action has been performed. The recalculated value is returned and is presented in the third column. Under the purchase and lease columns the equipment value is determined by the appraisal module 1305 using the new equipment sale price less a discount generated as a depreciation, de-install cost, and transaction cost for reselling the purchased equipment. For example, the value may be purchase price less 5-10% discount.

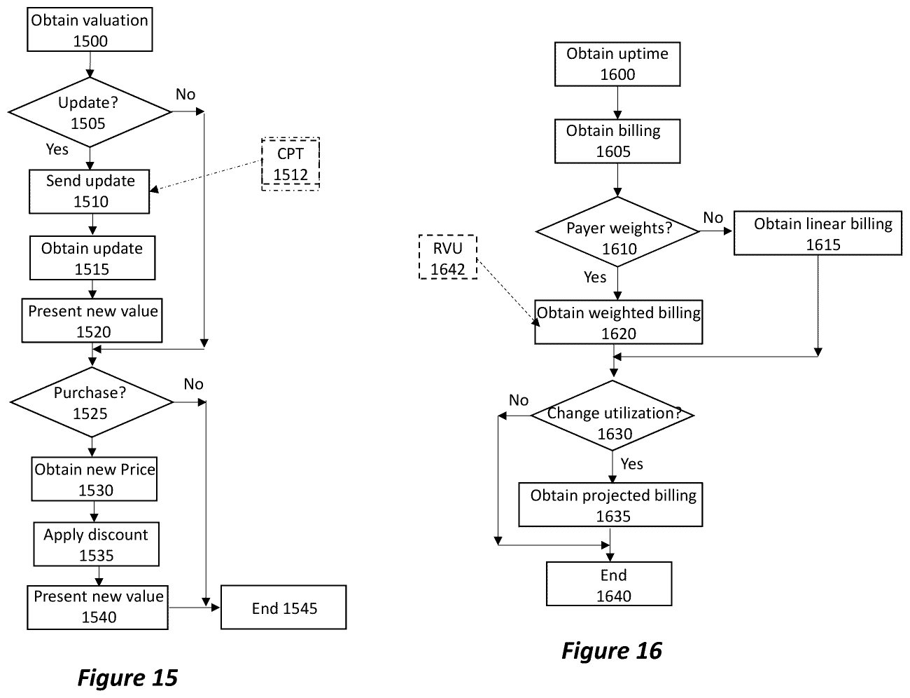

An example of a process that may be used is illustrated in FIG. 15. At 1500 the appraisal module generates an appraised valuation of the selected equipment. If an update/upgrade is selected at 105, the decision support module sends to the appraisal module at 1510 an indication of the update/upgrade selected. Then at 1515 the appraisal module sends to the decision support module a revised valuation which incorporate the value of the equipment with the new update/upgrade. At 1520 the decision support module presents the new appraised value. When at 1525 is a new purchase is selected, the price of the new equipment is obtained at 1530 either from a stored table of new equipment or by manual entry of the user. The a discount is applied to the purchase price at 1535 and the resulting new value is presented at 1540. The process ends at 1545.

The second row indicates the monthly volume of procedures performed. The value entered in the first column corresponds to data received from the equipment input module. Generally, every login to the equipment in order to perform a procedure is recorded by the equipment controller and is transmitted to the decision support module. The decision support module also logs the uptime and downtime of the equipment, as transmitted from the equipment's controller. For each piece of equipment, the decision support module 1300 is programmed with the time it takes to conventionally complete a procedure. Since the decision support module 1300 receives the uptime of the equipment and is programmed with the time it takes to perform a single procedure, it enables the user to enter a chosen value of forecasted number of procedures under the second column, and it thereafter calculates the financial consequences of such a forecasted change. For example, the user may enter 500 in the second column when it is determined that the equipment has sufficient uptime to support 500 procedures a month. This will increase the utilization of the equipment, in this example from 66% to 75%.

In this respect, whenever this disclosure refers to user selection or entry, this may be performed using the example illustrated in FIG. 14. That is, under each scenario, the user may click on the “Explore” button and a new window will open enabling the user to enter different variables.

The decision support module 1300 also receives data from the HR and accounting systems. Consequently, it displays the total amount of billings from the last period that were attributable to the selected equipment. In this example, in the last month the amount of billing is $128,305. Using this amount and the number of procedures performed, the decision support module 1300 calculates the predicted billing for the number of procedures entered by the user in the second column, here $146,467. This can be done linearly by dividing the total amount billed by the total number of procedures performed in order to obtain a billing per procedure. Conversely, this calculation can be performed using payer weighting. For example, the system may be preprogrammed with the amount per procedure that is agreed upon with each particular insurer/payer. Then the amount of the total billing is internally allocated according to the payers billed. Under such an example, the amount in the second column is calculated using the same ratio of payers as in the last period, i.e., the last month in this example. So, if, for example, the last month 30% was billed to Medicare, 40% to insurance company A, and 30% to insurance company B, the same ratio would be used to calculate the total amount in the second column under increased utilization.

An example for a process that may be used is illustrated in FIG. 16. At 1600 the uptime of the system for the last period (e.g., last month) is obtained, and in 1605 the total billing of procedures performed on the equipment for that same period is obtained. In 1610 it is determined whether weighted payer data is available. If no data is available, at 1615 the total billing is divided by the total number of procedures to obtain an average billing per procedure. Conversely, if weighting data is available, at 1620 the total billing is divided using the weights data to obtained a weighted average billing per procedure. At 1635 if a user enter a change in utilization, the billing per procedure is used to calculate projected total billing for the new utilization. The process ends at 1640.

The next row presents the expense for monthly maintenance cost, and in the first column it is the actual expense incurred in the last period, i.e., the last month. For the second column, the user may be provided with a choice to either use the same values as obtained for the last month, or increased by user selected amount or by the same ratio as the increase in the number of procedures. For example, when the equipment is relatively new, it may not require additional maintenance to perform additional procedures. Conversely, if the equipment is old and tends to break down after a given number of procedures, the maintenance amount may be increased.

The next row indicates the monthly operating costs, which generally include direct, indirect, fixed, and variable costs. For example, the costs include fixed costs such as the facilities housing the equipment and variable costs such as consumables necessitated by each procedure. The costs also include labor costs attributed to the operation of the equipment. The actual values are obtained from the HR module 1320. As noted elsewhere in this disclosure, the values received from HR relates to all labor costs, but the system extract the values attributable to each particular piece of equipment.

In the case of monthly operating costs, the amount for the second column under increased utilization can also be controlled by the user. The user may select to use the prior month costs, or increase the costs as needed for the increased utilization. For example, the facilities costs are fixed and would not change upon increase in utilization. Similarly, if two technicians were hired to operate the equipment and no additional technicians are needed to increase the utilization, then the costs would not increase. On the other hand, the user may decide that an additional technician would need to be hired to increase the utilization. The user may then increase the cost by the appropriate labor amount.

Using the above detailed information, the decision support module 1300 calculates the monthly gross profit for the last period and for the other scenarios presented on the user interface, as illustrated in FIG. 14. Additionally, using the variables described so far, the decision support module 1300 also calculates a break-even point (BEP). The BEP is represented as the number of procedures that need to be performed over a given period (say, one month) in order to cover the total operating cost of the equipment. In this case as well, the total operating cost includes the direct and indirect costs and fixed and variable costs.

Note that while in FIG. 14 the BEP under the increased utilization scenario is the same as that shown for the last month scenario. However, in some equipment the variable costs (e.g., consumables) for performing each procedure may increase upon increased utilization. Consequently, the calculated BEP may show an increase from last month. Also, for the pay-per-use case the BEP may be lower, since the company providing the equipment would normally carry much of the maintenance and carry the stock of consumables.

The next column provides financial predictions resulting from investment in the existing equipment. As illustrated by the callout in FIG. 14, when clicking on the explore button under the investment option, the user receives a drop-down menu with available options, e.g., refurbish the equipment, upgrade the hardware, upgrade the software, expand the capabilities of the equipment (e.g., enable new procedures not available previously), etc. Each option includes the cost to implement that option. When a user selects an option, the decision support module 1300 sends the selection to the appraisal module 1305 to recalculate the value of the equipment after the selected investment has been implemented. The new value is then presented under the investment column. For example, assuming that the user selected to invest in expanding the capabilities of the equipment, at a cost of $120,000. This selection is forwarded to the appraisal module for reevaluation of the equipment after implementation of the expansion. In this example, the appraisal module 1305 return a recalculated value of $350,000, which is lower than the initial value plus the cost of the upgrade, as there's reduction in value if the equipment were to be sold in the marketplace.

Upon completion of the expanded capability upgrade, the number of monthly procedures is estimated to increase to 538. In this particular example, although the number of procedures indicates an increase, since the capability has been expanded the utilization is actually decreased to 40%. This may be, for example, because it takes time to ramp up full utilization of the expanded capabilities of the equipment. Of course, this is not always the case, and the utilization may depend on the availability of trained technicians, the familiarity with the new capabilities, and with patients' demand for the new capabilities, etc.

Since an investment was made to upgrade the equipment, the break-even is increased to 190, to account for the cost of the upgrade and costs associated with the expanded capabilities. For example, the upgrade may require additional floor space, increase in electricity consumption, etc. In this example it would take 190 procedures per month to break even considering all of the fixed and variable operating cost of the equipment. Of course, with the increase in the number of procedures there's an increase in the amount of billing, which in the example of FIG. 14 shows $157,598. Also, the expanded capabilities require additional maintenance, such that maintenance costs increase to $12,197. Operating costs increase to $55,558, such that the gross comes to $89,843. Considering a five year at 5% discount rate, the net present value (NPV) of the resulting gross cash flow amounts to $4,547,678. Considering the increase of gross revenue resulting from the investment in the upgraded capabilities, the return on investment (ROI) is 461%. Also, the time for payback of the initial investment from the increased revenue is about 11 months.

The next column illustrates the predicted financial results in the case of replacing the old equipment with a purchase new machine. Here, the total cost of the new equipment may be about $3 million; however, it is shown that the value of new equipment is $2,775,770. Again, this is done by using the appraisal module 1305 to determine the value in the market place of a “just purchase” machine, which of course depreciates as soon as it is purchased by an original customer since, unless there's a shortage of supply, a secondary buyer would not pay a “new” price for a used equipment, even if it was just recently purchased.

The new machine may have expanded capabilities unavailable even with an upgrade of the old machine. Moreover, newer technology may have reduced the amount of time required for performing one procedure. Therefore, for this example it is assumed that the new machine would be capable of performing 650 procedures a month at a utilization of 70%. However, the new machine may require additional warranty payments and other increase in operating costs, so that the break-even point is raised to 204 procedures per month. Using linear relationship, the billing for the 650 procedures amount to $190,406. As noted, warranty payments may increase, such that the monthly maintenance costs may increase to $18,350 and operating costs may increase to $59,650. This results in a $130,756 gross income per month. Using the 5% discount rate for a five year return, the net present value is calculated to be $4,017,490 and the ROI over five years is 182%. The payback period is about 21 months. Note that in making the above calculations, the decision support engine 1300 includes an assumption that includes selling the old machine at the equipment value calculated by the appraisal module 1305, less the cost of reinstallation. That is, for example, the decision support module 1300 considers the cost of the new machine at the purchase price, less the sale of the old machine at the price estimated by the appraisal module 1305.

An alternative option presented to the user is a lease of a new machine. Again, all of the calculations for the lease option incorporate the revenue generated from the sale of the old equipment at the price provided by the appraisal module 1305. As shown by the second callout (dash-dot line), the explore button opens a menu that may include entries for the price of the new system, the down payment the user is prepared to pay, the term of the lease, and a residual value. The residual value can be calculated by the appraisal module 1305 by using the model of the system and calculating its value as a used system at the age equivalent of the lease term. Additionally, since lease payment are fully tax deductible, the user may enter the estimated tax rate of the organization for each of the lease term years.

Since it is a new system, the number of procedures and the utilization is similar to the entries from the purchase column. The break-even may be calculated by including or excluding the monthly lease payment. The monthly billing may be similar to that of the purchase column, although as noted previously the user may change the payers mix, such that the amount charged for each procedure may differ depending on the payer invoiced.

The maintenance costs may be included in the lease term, or the lease may exclude maintenance. The operating costs and the gross may be similar to purchase, in the case where the lease payments are excluded from the calculation.

As can be understood from this disclosure, the user may obtain predictive financial results to help in deciding how to handle existing medical equipment. Of course, there are other factors in making such decisions, but this disclosure provides a system that help simplifying at least the financial side of the decision making. Such a system is not currently available, such that the decision is made practically “in the dark” with respect to potential financial consequences.

Another feature illustrated in FIG. 13 is the publication modules: publication to internal market 1330 and publication to external market 1335. According to this embodiment, the user is provided the option to publish the specifically selected equipment for sale on an internal marketplace 1330, or external market place 1335. The system itself maintains an internal market place that is accessible only to users belonging to the same organization. For example, a particular health organization may have several hospitals in several states. It may be that a particular equipment is not sufficiently utilized in one hospital, but a different hospital within the same organization sees a need for such equipment. By enabling internal transfer within the organization, the organization can save all transaction costs associated with one hospital selling one equipment to a different organization, and then a different hospital purchasing the same equipment from an external source. Thus, the system enables a user to first publish the equipment for sale on the internal market place 130.

Notably, since all of the information regarding the equipment is already stored in the equipment database 1302 of the system, the publication is generated automatically using the data of the equipment available from the equipment database 1302 (age, maintenance, condition, etc.) and at the price generated by the appraisal module 1305. The user only has to click one button, “publish internally” and the new ad is generated automatically. The same is true for publishing to the external market place 1335, which is accessible to all users of the external marketplace 1335.

FIG. 13 also illustrates that the decision support engine 1300 receives data from the current procedural terminology (CPT) database 1303 code set, which provides medical, surgical, and diagnostic services and is designed to communicate uniform information about medical services and procedures among physicians, coders, patients, accreditation organizations, and payers for administrative, financial, and analytical purposes. This CPT is updated periodically according to publications by the American Medical Association. The CPT database 1303 also stores the relative value units (RVUs), which are a measure of value used in the United States Medicare reimbursement formula for physician services. However, as described above, the decision support engine can use the prior period data to calculate the cost per test for the organization and present that to the officers of the organization. The cost per test is not currently available to health organizations. Consequently, using the decision support engine calculation of the cost per test, the officers may now better negotiate the reimbursement amount that various insurers should pay for the various procedures, rather than simply rely solely on the RVUs. Moreover, as illustrated by the dashes lines in FIG. 16, the weighted billing may take into account the RVU corresponding to the procedures performed by the equipment.

Additionally, as illustrated in FIG. 15 by the dash-dot lines, when sending the information regarding the upgrade, the system may use the CPT code corresponding to the new procedures made possible using the new update/upgrade. Using this new CPT code, the decision support engine 1300 may calculate potential increase in utilization and provide a new procedure volume prediction. Similarly, the appraisal module 1305 can use the CPT code to update the appraise value of the updated/upgraded equipment. For new equipment purchase, the CPT codes that correspond to the procedures enabled by the new equipment would be uploaded to the CPT database.

Another feature of the decision support engine is providing a per modality financial calculations and predictions. For example, a user may select to perform analysis of all CT equipment operated by all of the facilities owned by the health organization. For this purpose, the decision support engine aggregates the financial performance values obtained for each equipment belonging to the selected modality. The financial decision engine can then use these values to perform predictive analytics. For example, based on the utilization the decision support engine can calculate how many units of certain equipment each hospital may need in the future. This prediction may be performed by also including data of demography and age of the population that may be served by the hospital. Similarly, by comparing maintenance costs of similar machines at different hospitals the decision support engine can indicate a specific machine that should be upgraded or replaced and, from that calculate predicted hardware budget for the future.

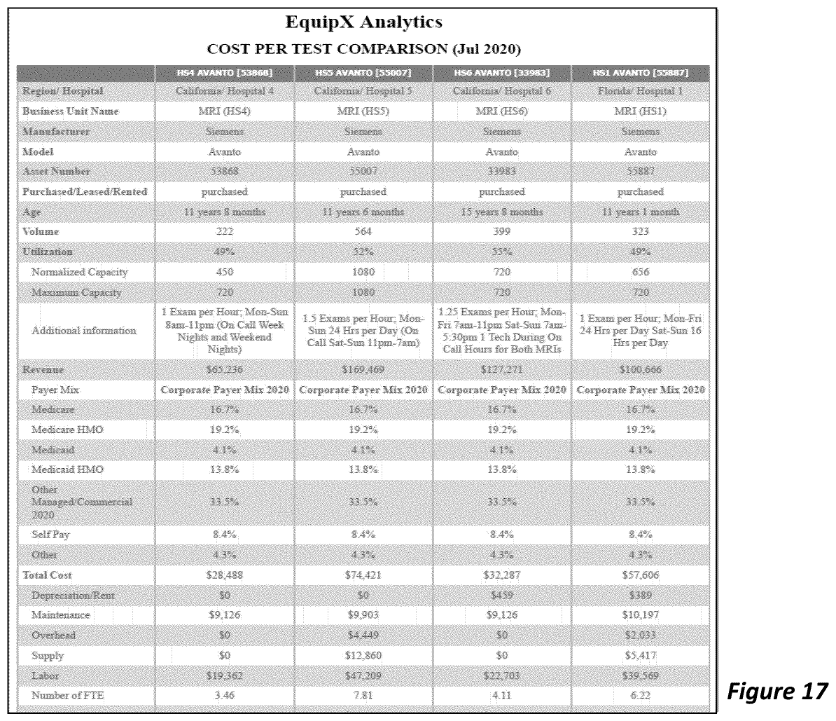

FIG. 17 illustrates an example of a screenshot for aggregated data on a per-modality analysis. In the example of FIG. 17 a cost per test comparison among the modality equipment operated by a particular health organization is presented. In this example, the organization owns four MRI machines, and so four columns are presented, one for each machine. The rows indicate relevant data regarding each of the machines, including whether it was purchased, leased, rented, etc. and the age of the machine, which is helpful in making upgrade/replacement decisions. The data also provides the actual utilization for prior period, and each machine's normal and maximum capacity. As with previous examples, the screenshot provides information regarding the revenue of each machine, and includes the breakdown of the payers, as each payer may be charged a different amount or have different payment terms for a particular test. This breakdown can be used when predicting future income upon consideration of future investments. Lastly, information regarding costs is provided, such as maintenance costs, overhead, supplies (consumables), and labor costs.

Thus, a system is disclosed for providing decision support regarding operation of medical equipment, the system comprising a processor and a memory storing executable instructions that, in response to execution by the processor, cause the system to at least: form a medical equipment database storing parameters data relating to medical equipment registered to the system; form an operation module that receives actual operating data of prior period, the operating data including number of procedures performed and operational costs; form an evaluation module, the evaluation module receiving the parameters data and the operating data and calculating therefrom a utilization value; form a decision support module receiving the parameters data and the utilization value corresponding to a subset of the medical equipment, the subset belonging to a selected modality, and generating a decision table on a user interface, the decision table providing aggregated data corresponding to operational condition of medical equipment corresponding to the selected modality.

The phrase “in one embodiment” is used repeatedly. The phrase generally does not refer to the same embodiment, however, it may. The terms “comprising”, “having” and “including” are synonymous, unless the context dictates otherwise. While the present invention has been related in terms of the foregoing embodiments, those skilled in the art will recognize that the invention is not limited to the embodiments described. The present invention may be practiced with modification and alteration within the spirit and scope of the appended claims. Thus, the description is to be regarded as illustrative instead of restrictive on the present invention.

While this invention has been discussed in terms of exemplary embodiments of specific materials, and specific steps, it should be understood by those skilled in the art that variations of these specific examples may be made and/or used and that such structures and methods will follow from the understanding imparted by the practices described and illustrated as well as the discussions of operations as to facilitate modifications that may be made without departing from the scope of the invention defined by the appended claims.

1. A computerized method for providing decision support engine for a user in medical equipment maintenance, comprising:
forming a decision support engine with a plurality of modules, including an appraisal module generating individualized market value for registered medical equipment, an impact analysis module determining profitability of each of the registered medical equipment, and a marketplace module enabling listing of the registered medical equipment;
generating a dashboard and presenting the dashboard on a computer monitor, the dashboard enabling the user to activate and interact with the various modules of the decision support engine;
forming an integration layer enabling data exchange between the decision support engine and the registered medical equipment;
storing in medical equipment database parameters data corresponding to the registered medical equipment, the parameters data including at least purchase price and purchase date;
using the dashboard to enter operational data corresponding to the registered medical equipment and using the operational data to generate the individualized market value for registered medical equipment by the appraisal module;
fetching by a decision support engine the parameter data and the appraised value of selected medical equipment, and fetching via a network billing information and expense information attributable to the selected medical equipment, and generating a decision table including operating results for a past period and estimated operating results predicted in view of upgrading the selected medical equipment and replacing the selected medical equipment with a new purchase or a lease.

2. The computerized method of claim 1, further comprising obtaining payroll data and calculating labor costs attributable to the selected medical equipment.

3. The computerized method of claim 1, further comprising operating the decision support module to calculate gross income attributable to the selected medical equipment.

4. The computerized method of claim 3, further comprising storing stores action items executable on the selected medical equipment, each of the action items includes an investment associated therewith wherein the action items include refurbish, hardware upgrade, software upgrade, and capability expansion;
upon selection by the user of one of the action items, operating the appraisal module to recalculate the appraised value of the selected equipment based on execution of the action item; and
operating the decision support engine to calculate estimation of utilization, billing and operating costs resulting from execution of the action item.

5. The computerized method of claim 3, further comprising upon selection by the user of a new medical equipment, operating the decision support module to calculate estimation of utilization, billing and operating costs resulting from purchase of the new medical equipment.

6. The computerized method of claim 5, wherein upon selection by the user of the new medical equipment:
operating the appraisal module to calculate residual value of the new medical equipment based on a term of a lease agreement; and,
operating the decision support engine to calculate estimation of monthly lease payments based upon the price and the residual value.

7. The computerized method of claim 6, wherein the marketplace module forms an internal marketplace accessible solely to users within selected organization and an external marketplace accessible to all users; and wherein upon selection by the user of a specified medical equipment, operating the marketplace module to assemble and publish an advertisement in at least one of the internal marketplace and external marketplace.

8. The computerized method of claim 7, further comprising assembling the advertisement by establishing asking price using the appraised value from the appraisal module and parameters data obtained from the medical equipment database.

9. A method for forming and operating a decision support engine regarding medical equipment, the method comprising the steps of:
form a medical equipment database storing parameters data relating to medical equipment registered to the system;
form an appraisal module, the appraisal module returning an appraised value of a selected medical equipment from the medical equipment database in response to data input of the medical equipment;
form a decision support module receiving parameters data corresponding to the selected medical equipment from the medical equipment database and receiving the appraised value from the appraisal module and fetching via a network connection billing data and cost data and generating a decision table corresponding to the selected equipment, the decision table comprising entry of the appraised value, entry of utilization calculated based on operating time of the selected medical equipment, entry of billing attributable to the selected medical equipment calculated from the billing data, and entry of operating cost attributable to the selected medical equipment calculated from the cost data.

10. The method of claim 9, wherein the decision support module further calculates gross income attributable to the selected medical equipment.

11. The method of claim 10, wherein:
the medical equipment database further stores action items executable on the selected medical equipment, each of the action items includes an investment associated therewith;
upon selection by a user of one of the action items, the appraisal module recalculate the appraised value of the selected equipment based on execution of the action item; and,
upon the selection by the user of one of the action items, the decision support module calculates estimation of utilization, billing and operating costs resulting from execution of the action item.

12. The method of claim 11, wherein the action items include refurbish, hardware upgrade, software upgrade, and capability expansion.

13. The method of claim 12, wherein the medical equipment database further stores price of new medical equipment and, upon selection by a user of one of the new medical equipment, the decision support module calculates estimation of utilization, billing and operating costs resulting from purchase of the new medical equipment.

14. The method of claim 13, wherein upon selection by a user of one of the new medical equipment, the decision support module calculates estimation of utilization, billing and operating costs resulting from lease of the new medical equipment.

15. The method of claim 14, wherein upon selection by a user of one of the new medical equipment:
the appraisal module calculate residual value of the new medical equipment based on a term of a lease agreement; and,
the decision support module calculates estimation of monthly lease payments based upon the price and the residual value.

16. The method of claim 15, wherein the method further forms an internal marketplace accessible solely to users within selected organization and an external marketplace accessible to all users; and wherein upon selection by the user the decision support module assembles and publishes an ad in at least one of the internal marketplace and external marketplace.

17. The computer-readable storage medium of claim 16, wherein the decision support module assembles the ad by establishing asking price using the appraised value from the appraisal module and parameters data obtained from the medical equipment database.
